(19) United States
(12) Patent Application Publication
(10) Pub. No.: US 2011/0166444 A1
(43) Pub. Date: Jul. 7, 2011

(54) MRI-VISIBLE SUTURES FOR MINIMALLY INVASIVE IMAGE-GUIDED ANASTOMOSIS

(75) Inventor: Daniel R. Elgort, New York, NY (US)

(73) Assignee: KONINKLIJKE PHILIPS ELECTRONICS N.V., EINDHOVEN (NL)

(21) Appl. No.: 13/061,820

(22) PCT Filed: Sep. 9, 2009
(86) PCT No.: PCT/IB09/53948

§ 371 Date: Mar. 2, 2011

Related U.S. Application Data
(60) Provisional application No. 61/098,807, filed on Sep. 22, 2008.

(51) Int. Cl. A61B 5/055; A61B 17/064

(52) U.S. Cl. 600/424

(57) ABSTRACT

When performing image-guided surgical procedures, a surgical fastener (19) (e.g., a suture or staple) is embedded or coated with an MRI-visible contrast agent or material. Depending on the type of MRI visible material (e.g., paramagnetic, superparamagnetic, diamagnetic, chemical shift, ultra-short echo time, etc.), a corresponding imaging sequence is selected and executed to generate contrast in an image of the surgical fastener. This image is presented to a user in real time, such as during an arthroscopic procedure, or may be generated and presented after a surgical procedure to image the fastener(s) and permit evaluation of the quality or success of the surgery.

The present innovation finds particular application in image-guided surgical procedures, particularly involving magnetic resonance imaging (MRI)-guided surgical procedures and the like. However, it will be appreciated that the described technique may also find application in other image-guided surgical systems, other surgical scenarios, other imaging techniques, and the like.

Anastomosis is a common part of many surgical procedures, in which two hollow organs (e.g., blood vessels or intestines) are sewn together following a resection or bypass of a diseased portion of the anatomy. When possible, it is desirable to perform surgical procedures using a minimally invasive, image-guided approach. Classical approaches use computed tomography (CT) guided procedures, which generate a radiation dose that is undesirable, especially for pregnant or pediatric patients.

Magnetic resonance imaging (MRI) is a desirable imaging modality for providing intra-operative guidance during minimally invasive interventions in which soft tissue contrast is critical and/or in patients that cannot tolerate ionizing radiation (e.g. pediatric or pregnant patients, etc.). Using MRI to guide surgical anastomosis is problematic because the sutures or staples that are used in the procedure are not visible in the MR images. This makes conventional MRI-guided surgical techniques ineffective for performing anastomosis and other surgical procedures or for using MRI to verify that the anastomosis or other surgical procedure was successful.

The present application provides new and improved systems and methods for MRI-guided anastomosis, which overcome the above-referenced problems and others.

In accordance with one aspect, a magnetic resonance visible fastener includes at least one of an MR contrast agent, an agent with MR-visible species, agents that generate a resonance frequency shift, an agent with a different resonance frequency from hydrogen, and an agent with an ultrashort echo time.

According to another aspect, a surgical fastener imaging system for magnetic resonance imaging (MRI)-guided surgical procedures includes one or more MRI-visible surgical fasteners, an MRI device that generates an image of the one or more MRI-visible surgical fasteners, and a sequence memory that stores a plurality of imaging sequences corresponding to different agents. The system further includes a control processor that selects an imaging sequence as a function of the type of contrast agent included in the one or more MRI-visible surgical fasteners and executes the selected imaging sequence to generate an image of the one or more MRI-visible surgical fasteners when imaging a patient during or after a surgical procedure employing the one or more MRI-visible surgical fasteners.

In accordance with another aspect, a method of imaging a subject during or after a surgical procedure to implant a surgical fastener includes selecting a magnetic resonance imaging (MRI) sequence to be executed as a function of a type of an MRI-visible agent included in the surgical fastener, executing the selected imaging sequence to generate MRI data, reconstructing an image of the subject and the surgical fastener, and presenting the image of the subject and the surgical fastener to a user.

One advantage is that the use of MRI to guide a surgical procedure is facilitated.

Another advantage resides in using MRI to evaluate surgical success and patient healing.

Still further advantages of the subject innovation will be appreciated by those of ordinary skill in the art upon reading and understand the following detailed description.

The innovation may take form in various components and arrangements of components, and in various steps and arrangements of steps. The drawings are only for purposes of illustrating various aspects and are not to be construed as limiting the invention.

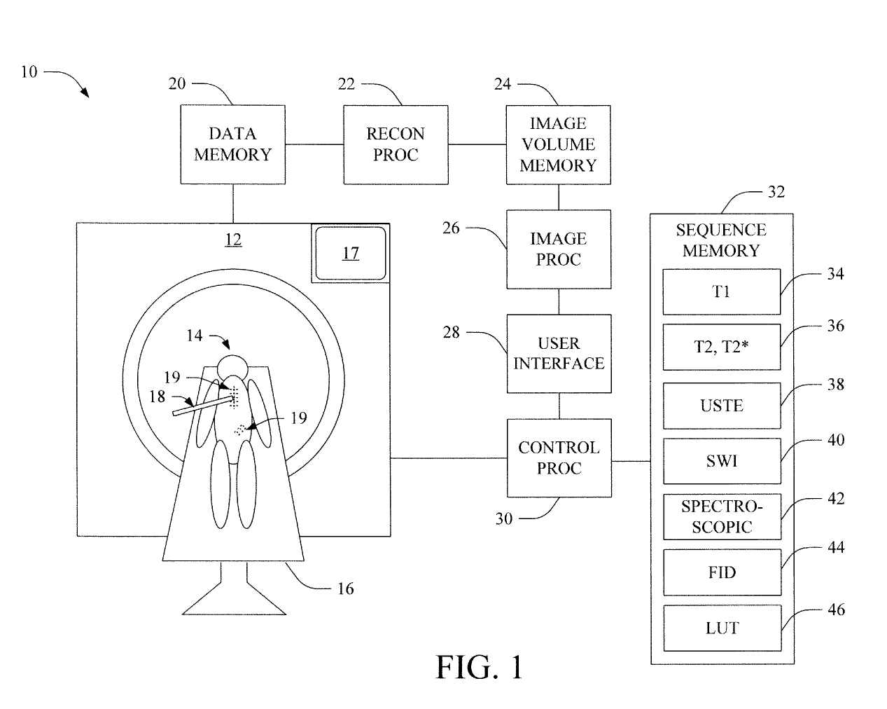

FIG. 1 illustrates a system that facilitates safely performing minimally invasive MRI-guided anastomosis or other surgical procedures (e.g., internal suturing or stapling, etc.) and enables MRI to be used to assess the success of the surgery post-operatively.

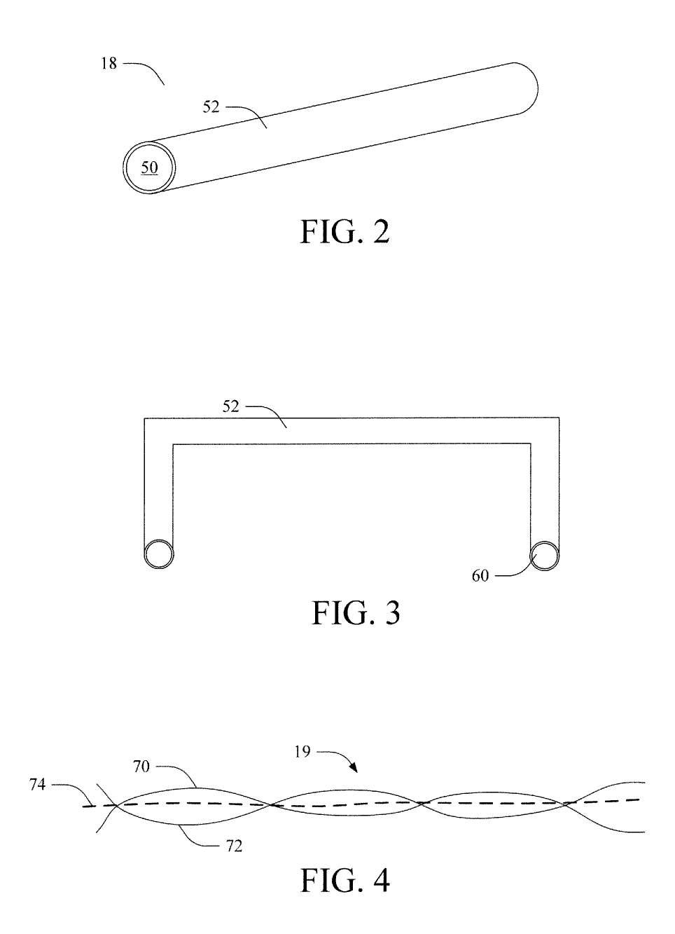

FIG. 2 illustrates a segment of a surgical fastener that includes a suture material that is coated with or includes a contrast agent or molecular material.

FIG. 3 illustrates an example of a surgical fastener that includes a surgical staple that is coated with a contrast agent or molecular material.

FIG. 4 illustrates an example of a surgical fastener that includes a multi-strand suture comprising suture strands that are interwoven or braided with an MRI-visible strand.

FIG. 1 illustrates a system 10 that facilitates safely performing minimally invasive MRI-guided anastomosis or other surgical procedures (e.g., internal suturing or stapling, etc.) and enables MRI to be used to assess the success of anastomosis post-operatively. By incorporating an MR-contrast agent (e.g. gadolinium-DTPA or the like) or an MR-visible molecular species that is not present in high concentrations in biological tissue (e.g. Fluorine), an MR imaging method is provided that generates MR images in which surgical fasteners are clearly visible. In other embodiments, an MR-visible coating or material is incorporated into or onto non-metallic medical devices or implants, prosthetics, fasteners, etc.

The system 10 includes an MRI device 12 in which a patient 14 is positioned on a patient support or table 16. An MR data acquisition scan generates an image of the interior of the patient that is displayed on a monitor 17 for the surgeon to use during the minimally invasive procedure. An insertable arthroscopic surgical device 18 that carries one or more tools, including a tool for stapling or suturing, is inserted into the patient. After the arthroscopic device is inserted a short distance, an MR tracking scan is conducted to locate the tip of the arthroscopic device relative to the patient's internal anatomy. Tracking techniques that are not MR based are also contemplated. The tracking scans are repeated regularly as the arthroscopic device moves to the surgical site. During the procedure, updated MR images are generated to monitor the progress. The tool inserts staples or sutures 19 (shown in dashed lines to indicate that they are internal to the subject) inserted by the tool are MR visible. Thus, as the tool moves along tissues to be joined, the sutures or staples are displayed in the MR images. For example, when attaching two tubular structures, e.g. intestines, blood vessels, etc., the MR images show whether the sutures or staples completely encircle the tubular structures. After the surgery, the arthroscopic device is removed and the entry point closed. The MR-visible sutures 19 that are also visible subsequent MR imaging sessions to evaluate post-operation success, healing progress, and/or quality of the sutures or other fasteners.

More specifically, the system 10 includes a data memory 20 that is coupled to the MRI data acquisition device 12 and that stores raw MR image data. The data memory is coupled to a reconstruction processor 22 that reconstructs an image volume representation of the patient or a volume of interest. Volume image data is then stored in a volume image memory 24, and an image volume processor 26 employs the MR image volume data to generate an MR image that is presented to an operator on the display. A user interface 28, which in one embodiment includes the monitor 17, is coupled to the image processor to select images or portions of images to be displayed, image filtering, enhancement or other processing to be conducted, and the like. The interface is also coupled to a control processor 30, which activates the MRI device according to a desired or predefined imaging sequence stored in a sequence memory 32. The sequence memory includes, for instance, T1 imaging sequence(s) 34, T2 imaging sequence(s) 36, ultra-short echo time (USTE) imaging sequences 38, susceptibility-weighted imaging sequences 40 (e.g., high-spatial-resolution 3D gradient-echo MR imaging sequences or techniques), spectroscopic imaging sequences 42, and any other suitable imaging sequences for imaging the various and/or specific MR-visible materials described herein. Moreover, the control processor can selectively alternate through one or more imaging sequences when, for instance, multiple imaging sequences are desired to image sutures or other medical objects having different MR-visible coatings, materials, or characteristics.

In one embodiment, surgical fasteners such as sutures or staples incorporate an MR-visible chemical agent and the MR image acquisition/reconstruction method is configured to generate contrast between the fasteners and the surrounding anatomy, thereby facilitating visualizing the surgical fasteners within MR images (e.g., either in real-time intra-procedural images used for interventional guidance, or in post-operative images used for follow-up assessment of the procedure's success or quality).

The fasteners are constructed by embedding or coating standard surgical sutures, staples, or the like, with an MR contrast agent or an MR-visible molecular species that is not present in high concentrations in biological tissue. The MR-visible agents or molecules include without being limited to: paramagnetic contrast agents (e.g., Gadolinium-DTPA); superparamagnetic contrast agents (e.g., iron oxide nanoparticles); diamagnetic agents (e.g., barium sulphate); an agent with a large chemical shift relative to the hydrogen atoms in water and fat molecules (e.g., acetic acid); an agent that generates an MRI signal at a distinct frequency from the hydrogen atoms (e.g., a molecular species that includes Fluorine); an agent with an ultra-short TE (e.g., rubber filaments); etc.

The MR image acquisition and reconstruction technique is configured to generate contrast between the fasteners and/or implants and the surrounding anatomy and depends on the type of agent that is incorporated into the fastener and/or implants. For instance, if a paramagnetic agent is used, T1-weighted images are acquired using the T1 imaging sequence(s) 34 and reconstructed to create contrast between the fastener and/or implants and the surrounding anatomy. If a superparamagnetic agent is used, T2-weighted, T2*-weighted, or susceptibility-weighted images are acquired using the T2 imaging sequence(s) 36 or SWI imaging sequence(s), respectively, and reconstructed to create contrast between the fastener and/or implants and the surrounding anatomy. If a diamagnetic agent is used, T2-weighted, T2*-weighted, or susceptibility-weighted images are acquired, using the T2 imaging sequences 36 (which includes T2 and T2* imaging sequences) and/or SWI sequences 40, and reconstructed to create contrast between the sutures and the surrounding anatomy.

In another embodiment, if an agent with a large chemical shift (relative to the hydrogen atoms in water or fat molecules) is used, imaging is performed using a narrow-band off-resonance excitation pulse or spectroscopic imaging technique or algorithm 42 to create contrast between the sutures and the surrounding anatomy.

In another embodiment, when using an agent that generates an MRI signal at a distinct frequency different from the frequency of hydrogen atoms, imaging is performed using RF excitation pulses that are tuned to the resonant frequency of that agent, and RF receive coils in the MRI device 12 are also tuned to agent's resonant frequency.

In yet another embodiment, if an agent with an ultra-short TE is used, a free induction decay (FID) imaging sequence 44 with an ultra-short echo time is used to create contrast between the fastener and/or implants and the surrounding anatomy.

Additionally, the fasteners or sutures are biocompatible so that the contrast agent or molecular coating readily bonds to the surface thereof. In another embodiment, the sutures are biodegradable and disintegrate in the patient after a period of time.

In this manner, the described systems and methods enable minimally invasive MRI-guided anastomosis and other surgical procedures to be performed safely, and enable MRI to be used to assess the success of anastomosis post-operatively. This feature facilitates performing many clinically relevant surgical procedures, including minimally invasive MR-guided pediatric procedures, MR-guided gastrointestinal resection, MR-guided coronary artery bypass, MR-guided urology procedures (e.g., radical prostatectomy), MR-guided microsurgical procedures (e.g., tubal ligation or vasectomy), MR-guided in-utero surgical procedures (e.g., in-utero surgery to close a persistent foramen ovale in a fetal heart, etc.).

According to an example, a user (e.g., a surgeon or technician) selects a fastener having a known MRI-visible material (e.g., a contrast agent embedded in or coated on the fastener) for a surgical procedure. The user indicates to the system, via the user interface 28, the type of MRI-visible material in or on the fastener, and the control processor 30 executes a table lookup algorithm on a lookup table (LUT) 48 in the sequence memory 32 to identify an appropriate imaging sequence to be executed when imaging the subject. During the procedure, which may be a robotic surgical procedure or may be performed by a human), the MRI device 12 is used to image the subject as the surgical fasteners are employed to fasten tissue in the subject, and the surgical fasteners are visible.

In another example, a user indicates to the system, via the user interface, the identity of the type of MRI-visible material in or on the fastener(s) at some time after a surgical procedure (e.g., anastomosis, etc.), and the control processor looks up an appropriate imaging sequence to execute when imaging a patient positioned in the MRI device in order to generate an image of the fasteners to evaluate the success or quality of a prior surgical procedure. In another embodiment, the post-operative imaging procedure is used to image a surgical implant (e.g., a stent, a stainless steel fastener such as a screw or staple an artificial joint part, a pacemaker, and/or its leads, etc.) that includes or is coated by an MRI-visible material.

FIG. 2 illustrates a segment of a surgical fastener 19 that includes a suture material 50 that is coated with or includes a contrast agent or molecular material 52. For instance, the suture material may include or be coated with a superparamagnetic agent, and one or more of T2-weighted, T2*-weighted, or susceptibility-weighted images are acquired using the T2 imaging sequence(s) 36 and/or SWI imaging sequence(s), respectively. The image is then reconstructed to create contrast between the fastener and/or implants and the surrounding anatomy.

In another embodiment, one or multiple contrast agents are provided in or on the fastener material, and corresponding imaging sequences are used to image the fastener. For instance, particles of a diamagnetic agent and a paramagnetic agent can be embedded or suspended in a plastic fastener to make the fastener MRI-visible. In this example, a T2*-weighted sequence or susceptibility-weighted image of the diamagnetic material is generated using the T2* image sequence 36 and/or the susceptibility imaging sequence 40, and the paramagnetic material is imaged using T1 imaging sequence(s) 34. MR-imagable molecules or materials can be embedded in, bound to, suspended in, encapsulated in, etc., plastic polymer chains, plastic lattice structures, plastic sheet structures such as branched polymer chains, etc., that are used to coat or form the fastener. In another embodiment, the MRI-visible material is deposited onto a plastic substrate to which it bonds (e.g., chemically, mechanically, electrostatically, etc.), and the plastic substrate is used to coat or form the fastener.

FIG. 3 illustrates an example of a surgical fastener 19 that includes a surgical staple 60 that is coated with a contrast agent or molecular material 52. For instance the staple 60 can be formed of stainless steel and the molecular material includes a plastic coating that includes a contrast agent. In another embodiment, the coating includes multiple contrast agents or materials that are imaged using one or more imaging sequence types. For instance, the coating may include a paramagnetic agent that is imaged using T1 imaging sequences and a superparamagnetic agent that is imaged using T2 sequences, etc.

FIG. 4 illustrates an example of a surgical fastener 19 that includes a multi-strand suture comprising suture strands 70 and 72 that are interwoven or braided with an MRI-visible strand 74. For instance, a thread or strand 74 including or coated with an MRI-visible material (e.g., a paramagnetic contrast agent, a superparamagnetic contrast agent, a diamagnetic contrast agent, a large chemical shift agent, an agent with a distinguishable frequency, an ultra short TE agent, etc.) can be interwoven or braided into the suture filaments 70, 72. In another embodiment, one or more different MRI-visible agents are incorporated into the fastener 19 and imaged using one or more corresponding imaging sequences.

It will be appreciated that the systems and methods described herein are not limited to making surgical fasteners visible to MRI devices, but rather are applicable to any instrument or device (e.g., surgical implants or devices, stents, electrical leads, pacemakers, brain stimulators, etc.) for which MRI visibility is desirable.

The innovation has been described with reference to several embodiments. Modifications and alterations may occur to others upon reading and understanding the preceding detailed description. It is intended that the innovation be construed as including all such modifications and alterations insofar as they come within the scope of the appended claims or the equivalents thereof.

1. A magnetic resonance visible fastener including at least one of:
an MR contrast agent, an agent with MR-visible species, agents that generate a resonance frequency shift, an agent with a different resonance frequency from hydrogen, and an agent with an ultrashort echo time.

2. The fastener according to claim 1, wherein the contrast agent includes at least one of:
a paramagnetic contrast agent, for example gadolinium DTPA;
a superparamagnetic contrast agent, for example iron oxide nanoparticles.

3. The fastener according to claim 1, including:
a surgical suture that is one of coated with, incorporated with, interwoven with, bonded to, or embedded with, the agent, wherein the agent is at least one of:
a diamagnetic agent, for example barium sulphate;
a paramagnetic contrast agent, for example gadolinium DTPA;
a superparamagnetic contrast agent, for example iron oxide nanoparticles.

4. The fastener according to claim 1, including:
a surgical staple which is one of coated with, incorporated with, bonded to, or embedded with, the agent, wherein the agent is at least one of:
a diamagnetic agent, for example barium sulphate;
a paramagnetic contrast agent, for example gadolinium DTPA;
a superparamagnetic contrast agent, for example iron oxide nanoparticles.

5. A surgical fastener imaging system for magnetic resonance imaging (MRI)-guided surgical procedures, including:
one or more MRI-visible surgical fasteners according to claim 1;
an MRI device that generates an image of the one or more MRI-visible surgical fasteners;
a sequence memory that stores a plurality of imaging sequences corresponding to different agents; and
a control processor that selects an imaging sequence as a function of the type of contrast agent included in the one or more MRI-visible surgical fasteners and executes the selected imaging sequence to generate an image of the one or more MRI-visible surgical fasteners when imaging a patient during or after a surgical procedure employing the one or more MRI-visible surgical fasteners.

6. The system according to claim 5, wherein the agent includes a paramagnetic contrast agent and the control processor selects and executes a T1 imaging sequence and the MRI device generates T1-weighted image data that is reconstructed into an image of the one or more surgical fasteners by a reconstruction processor and presented to a user on a user interface.

7. The system according to claim 5, wherein the agent includes a superparamagnetic contrast agent, such as iron oxide nanoparticles and the control processor selects and executes at least one of a T2 or T2* imaging sequence or a susceptibility-weighted imaging sequence, and the MRI device generates T2-, T2*-, or susceptibility-weighted image data, respectively, that is reconstructed into an image of the one or more surgical fasteners by a reconstruction processor and presented to a user on a user interface.

8. The system according to claim 5, wherein the agent includes a diamagnetic agent, such as barium sulphate, and the control processor selects and executes at least one of a T2 or T2* imaging sequence or a susceptibility-weighted imaging sequence, and the MRI device generates T2-, T2*-, or susceptibility-weighted image data, respectively, that is reconstructed into an image of the one or more surgical fasteners by a reconstruction processor and presented to a user on a user interface.

9. The system according to claim 5, wherein the agent has a detectable chemical shift relative to hydrogen atoms in fat tissue or water, such as acetic acid, and the control processor selects and executes a spectroscopic imaging sequence, and the MRI device generates spectroscopic data that is reconstructed into an image of the one or more surgical fasteners by a reconstruction processor and presented to a user on a user interface.

10. The system according to claim 5, wherein the agent generates an MR signal at a frequency different from that of hydrogen atoms in fat tissue or water, such as fluorine, wherein the MRI device generates image data by emitting an RF excitation pulse tuned to the tuned to the frequency of the contrast agent and detected by RF coils tuned to the frequency of the contrast agent, and wherein the image data is reconstructed into an image of the one or more surgical fasteners by a reconstruction processor and presented to a user on a user interface.

11. The system according to claim 5, wherein the agent has an ultra-short echo time, the control processor selects and executes a free induction decay imaging sequence with an ultra-short echo time, and the MRI device generates image data that is reconstructed into an image of the one or more surgical fasteners by a reconstruction processor and presented to a user on a user interface.

12. The system according to claim 5, wherein the surgical procedure includes an arthroscopic procedure connecting tubular anatomical structures.

13. A method of imaging a subject during or after a surgical procedure to implant the surgical fastener according to claim 1, the method including:
selecting a magnetic resonance imaging (MRI) sequence to be executed as a function of a type of the agent included in the surgical fastener;
executing the selected imaging sequence to generate MRI data;
reconstructing an image of the subject and the surgical fastener; and
presenting the image of the subject and the surgical fastener to a user.

14. A method of imaging a subject during or after a surgical procedure to implant a surgical fastener, including:
selecting a magnetic resonance imaging (MRI) sequence to be executed as a function of a type of an MRI-visible agent included in the surgical fastener;
executing the selected imaging sequence to generate MRI data;
reconstructing an image of the subject and the surgical fastener; and
presenting the image of the subject and the surgical fastener to a user.

15. The method according to claim 14, wherein the surgical object is at least one of a surgical suture or a surgical staple having the contrast agent embedded therein or coated thereon.

16. A fastener for use in the method according to claim 14.
